(19) United States
(12) Patent Application Publication
(10) Pub. No.: US 2007/0286908 A1
(43) Pub. Date: Dec. 13, 2007

(54) Salt and soap compositions

(76) Inventor: Kent Clampitt, Laguna Hills, CA (US)

Correspondence Address: STOUT, UXA, BUYAN & MULLINS LLP, 4 VENTURE, SUITE 300, IRVINE, CA, 92618, US

(21) Appl. No.: 11/811,185

(22) Filed: Jun. 7, 2007

Related U.S. Application Data
(60) Provisional application No. 60/812,673, filed on Jun. 8, 2006.

(51) Int. Cl. A61K 33/14; A61K 36/889; A61K 36/254; A61K 36/82; A61K 36/54; A61K 36/53; A61K 36/8962; A61K 36/31; A61K 36/906; A61K 36/48; A61K 36/258

(52) U.S. Cl. 424/680; 424/195.15; 424/195.17; 424/725; 424/757; 424/755; 424/756; 424/729; 424/764; 424/765; 424/745; 424/778; 424/777; 424/727; 424/739; 424/728; 424/754

(57) ABSTRACT

New compositions including salt components and/or antioxidant-containing botanical extract components, such as bamboo leaf extracts, acai berry extracts, lotus leaf extracts, other antioxidant-containing botanical extracts and mixtures thereof, are provided. The present compositions effectively take advantage of the chemical make-ups and/or properties of these components to provide new and/or enhanced benefits relative to substantially identical compositions without such components.

### RELATED APPLICATION

This application claims the benefit of U.S. Provisional Application No. 60/812,673 filed Jun. 8, 2006, the disclosure of which is hereby incorporated by reference in its entirety herein.

### BACKGROUND OF THE INVENTION

The present invention generally relates to compositions including antioxidant-containing botanical extracts. More particularly, the invention relates to compositions, such as salt compositions, skin care compositions, bathing compositions, soap compositions and the like including one or more of such extracts.

Salt, for example, sodium chloride, is used to season/flavor food. Various salt-based compositions are available as food additives. Substantially pure sodium chloride is a reasonably adequate seasoning/flavor enhancer for food. In addition, sea salt, that is salt derived from sea water, often provides a somewhat different flavoring to food.

Skin care products and soaps often include oils including fragrance oils and the like which give such products and soaps a pleasant fragrance.

Various botanical extracts include antioxidants, and often other substances, which may be of value. For example, and without limitation, bamboo leaf extract is rich in antioxidants and other active compounds, including flavones, phenolic acid, lactone, polyose, amino acid, and microelements. Such extract has been suggested for use in dietary supplements, and in drug, food, anti-aging and cosmetic products.

Acai berry extract is rich in antioxidants and other active compounds, including polyphenols and flavonids, mostly of the anthocyanin and proanthocyanin categories. Such extract has been suggested for use in dietary supplements, and in drug, food, anti-aging and cosmetic products.

Lotus leaf is a rich source of antioxidant flavonids. It contains nuciferine, N-nonuciferine, O-nornucifeine, anonaine, armepavine, N-methyt-coclaurine, paronuciferine, liriodenine, dehydro-roemerine, nelumboside, quercetin and isoquercetin. Such extract has been suggested for use in dietary supplements, and in drug, food, anti-aging and cosmetic products.

It would be advantageous to provide new salt-containing products, new skin care products and new soaps which, for example, provide new and/or enhanced benefits to humans using such new products.

### SUMMARY OF THE INVENTION

New compositions including salt components and/or extract components, in particular antioxidant-containing or antioxidant-rich botanical extract components have been discovered. The present compositions effectively take advantage of the chemical make-ups and/or properties of these components to provide new and/or enhanced benefits relative to substantially identical compositions without such components. For example, compositions comprising both a salt component such as a sea salt component, and an antioxidant-containing botanical extract component, such as noted herein, provide one or more new and/or enhanced benefits, such as flavor, taste, aroma, nutritional value, therapeutic value and the like, which are present and/or are enhanced because of the presence of both of these components. In addition, new compositions, such as skin care compositions and soap compositions, including one or more of the antioxidant-containing botanical extract components as described herein, with or without a salt component, are within the scope of the present invention. Such compositions, for example, as described elsewhere herein, advantageously provide new and/or enhanced benefits relative to substantially identical compositions without the extract components.

The present compositions are relatively easy to produce, for example, employing mixing and/or blending equipment and techniques which are conventional and well known in the art, and can easily and effectively be used, for example, in ways that are substantially similar to conventional products of the same general types.

In one broad aspect, the present invention is directed to salt compositions comprising a sea salt component and an antioxidant-containing botanical extract component combined with the sea salt component. The extract component is advantageously present in an amount effective to provide the composition with at least one of (a) a taste, (b) an aroma, (c) a color, (d) a nutritional value and (e) a therapeutic value. Such benefits (a), (b), (c), (d) and (e) are relative to a substantially identical composition without the extract component. Thus, the present salt compositions not only have the advantage of a sea salt component but further have enhanced flavor, smell or aroma, color, nutritional value and/or therapeutic value as a result of the presence of the extract component.

Any suitable antioxidant botanical extract or mixtures thereof can be used in accordance with the present invention provided that the extract or extracts are compatible with the other materials in the composition and are effective without resulting in substantial or significant deleterious effect or effects, for example, to the human using or handling the composition.

Examples of useful antioxidant-containing botanical extract components include, without limitation, a bamboo leaf extract component, an acai berry extract component, a lotus leaf extract component, a pitaya extract component, a green tea extract component, a red tea extract component, a Reishi mushroom extract component, a Shitake mushroom extract component, a licorice root extract component, a burdock root extract component, a soy extract component, a carrot extract component, a tomato extract component, a broccoli extract component, a Chlorella extract component, a wheatgrass extract component, a Laminaria Digitata extract component, an alfalfa extract component, a Rose Hips extract component, a ginger extract component, a clove extract component, a cinnamon extract component, an oregano extract component, a thyme extract component, a fennel extract component, a sage extract component, a rosemary extract component, a turmeric extract component, a garlic extract component, an onion extract component, a ginseng extract component, an Astragalus extract component, a Schizandra extract component, a Fenugreek extract component, and mixtures thereof.

In a useful embodiment, the antioxidant-containing botanical extract component is selected from a bamboo leaf extract component, an acai berry extract component, a lotus leaf extract component and mixtures thereof.

In a useful embodiment, the extract component is present in an amount in a range of about 1.0 or less to about 5.0 or about 10 or about 30 or more milliliters per kilogram of the sea salt component.

In one embodiment, the composition includes a bitterns component in an amount effective to provide the composition with a taste different from a substantially identical composition without the bitterns components. The bitterns component may be provided in the composition separately or separate from the sea salt component. At least a portion of the bitterns component, or even all the bitterns component, may be provided in the composition as part of the sea salt component.

The bitterns component may be present in the composition in an amount in a range of about 1.0 or about 2.0 to about 12 or about 20 or more milliliters per kilogram of sea salt component.

The sea salt component may be any salt component derived from sea water, for example, by heating, evaporation and the like processing. The sea salt component advantageously is selected from Pacific sea salt, sea salts substantially similar to Pacific sea salt, full-bodied sea salts, sea salts containing bitterns, dead sea salt and mixtures thereof.

In one embodiment, the present compositions further comprise an aqueous component in an amount effective to perform at least one of (1) facilitate the formation of the composition; (2) provide a bitterns component to the composition and (3) provide a taste to the composition. The aqueous component may be chosen from any suitable aqueous component. In a useful embodiment, the aqueous component is selected from fresh water, sea water, concentrated sea water and mixtures thereof. The aqueous component may be present in a very relatively broad range of amounts. In one embodiment, the aqueous component of the composition is present in a range of about 1.0 to about 20 milliliters per kilogram of the sea salt component.

The present compositions may further comprise a spice component, different from the sea salt component, in an amount effective to provide the composition with at least one of (a) a different taste, (b) a different color and (c) a different aroma relative to a substantially identical composition without the spice component.

In another broad aspect of the present invention, a composition useful in bathing at least a portion of a human body is provided. The composition comprises a salt component and an antioxidant-containing botanical extract component, for example, as described elsewhere herein, combined with the salt component. The composition is effective in facilitating the bathing of at least a portion of a human body in water including an effective amount of the composition. In one useful embodiment, the extract component is present in an amount effective to provide the composition with at least one of (a) an aroma, (b) a color and (c) an ability to provide a benefit or an enhanced benefit to at least a portion of the human body bathed in water including an effective amount of the composition.

In a further broad aspect of the present invention, skin care compositions are provided and comprise a base oil component, and an antioxidant-containing botanical extract component, for example, as described elsewhere herein, combined with the base oil component. The base oil component is present in an amount effective to provide at least one benefit to the skin region of a human to which the composition is applied.

The composition advantageously has the extract component present in an amount effective to provide the composition with at least one of (a) an aroma, (b) a color, and (c) an ability to provide a benefit or an enhanced benefit to a skin region of a human to which the composition is applied different from a substantially identical composition without the extract component.

In one embodiment, the base oil composition is a major amount by weight of the composition.

In one embodiment, the extract component is present in an amount in a range of about 0.5 or about 0.1 to about 0.5 or about 1 milliliter per 100 grams of the base oil component.

The base oil component may include any one or more of a large number of oil suitable for the present purpose. For example, and without limitation, such base oil component may comprise a material selected from the group consisting of beeswax, olive oil, avocado oil, jojoba oil, macadamia oil, aloe vera gel, rosemary essential oil extract and mixtures thereof.

In one useful embodiment, the present compositions comprise at least one other or additional component, for example, different from the extract component, present in an amount effective to provide the composition with at least one of (a) an aroma, (b) a color and (c) an ability to provide a benefit or an enhanced benefit to a skin region of a human to which the composition is applied different from a substantially identical composition without the other or additional component. Examples of such other or additional components which may be included in the present compositions include, without limitation, vitamins, lavender oil, rosemary extract, lemon oil, other citrus oils, palm oil, palmarosa oil, tea leaf oil, fragrance oils and the like and mixtures thereof. The other or additional components may be present in the present compositions in effective amounts for example, in an amount in a range of about 0.02 or about 0.05 to about 0.5 milliliters per 100 grams of the base oil component.

In one embodiment, the present compositions are substantially anhydrous.

In one embodiment, the present compositions comprise an aqueous component. The aqueous component may comprise a major amount of the composition.

In a useful embodiment for application to the skin, the present composition may be in the form of a balm, for example, a substantially anhydrous balm.

In an additional use embodiment, the compositions may be in the form of a cream, for example, a cream including an aqueous component.

In a still further broad aspect of the invention, body and hair soap compositions are provided which comprise a base oil component; a caustic soda component; a sea water component and an antioxidant-containing botanical extract component, for example, as described elsewhere herein; and another or an additional component, different from the extract component, for example, selected from essential oils, fragrance oils and mixtures thereof.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

### DETAILED DESCRIPTION

Salt compositions in accordance with the present invention generally comprise a salt or sodium chloride component, advantageously, a sea salt component, and an antioxidant-containing botanical extract component, for example, as described elsewhere herein, combined with the sea salt component.

In one embodiment, the extract component is present in the composition in an amount effective to provide the composition with at least one of a different and/or enhanced (a) taste, (b) aroma, (c) color, (d) nutritional value and (e) therapeutic value, for example, relative to a substantially identical composition without the extract component.

Examples of useful antioxidant-containing botanical extract component include, without limitation, a bamboo leaf extract component, an acai berry extract component, a lotus leaf extract component and mixtures thereof. In a useful embodiment, the antioxidant-containing botanical extract component is selected from a bamboo leaf extract component, an acai berry extract component, a lotus leaf extract component, a pitaya extract component, a green tea extract component, a red tea extract component, a Reishi mushroom extract component, a Shitake mushroom extract component, a licorice root extract component, a burdock root extract component, a soy extract component, a carrot extract component, a tomato extract component, a broccoli extract component, a Chlorella extract component, a wheatgrass extract component, a Laminaria Digitata extract component, an alfalfa extract component, a Rose Hips extract component, a ginger extract component, a clove extract component, a cinnamon extract component, an oregano extract component, a thyme extract component, a fennel extract component, a sage extract component, a rosemary extract component, a turmeric extract component, a garlic extract component, an onion extract component, a ginseng extract component, an Astragalus extract component, a Schizandra extract component, a Fenugreek extract component, and mixtures thereof.

In a useful embodiment, the antioxidant-containing botanical extract component is selected from a bamboo leaf extract component, an acai berry extract component, a lotus leaf extract component and mixtures thereof.

In a useful embodiment, the extract component may be present in an amount in a range of about 1.0 or less to about 5.0 or about 10 milliliters or more per kilogram, or about 0.1 or less to about 0.5 or about 1.0 or more teaspoons per pound, of salt component.

The antioxidant-containing botanical extract component, for example, and without limitation, bamboo leaf extract component, acai berry extract component and lotus leaf extract component, may be used separately, or as mixtures of two or three or more of such extract components. When the extract components are used together, each such component may be present in a variety of concentrations relative to concentrations of the other component or components. For example, each of the extract components may be present in an amount of about 5% by weight or less or about 10% by weight or about 20% by weight or about 50% by weight to about 60% by weight or about 80% by weight or about 90% by weight or about 95% by weight or more of the total extract components, for example, and without limitation, the total bamboo leaf extract component, acai berry extract component and lotus leaf extract component, present in the composition.

The present antioxidant-containing extract components may be obtained by conventional extraction processing of the desired botanical or portion thereof. The extract may include an aqueous carrier or a non-aqueous carrier. Many of the extract components described herein are commercially available. Conventional mixing techniques can be used to produce mixtures of extracts, as desired.

Any suitable salt component, advantageously sea salt component, may be used in the present compositions. Such salt component may include a major amount, that is at least about 50%, by weight of sodium chloride, for example, at least about 70% or about 90% or about 95% or more by weight of sodium chloride. In certain embodiments of the present invention, all or a portion of the salt component may comprise other salts, such as Epson salt and the like. The salt component may be derived from any suitable source using any suitable, for example, conventional and well known, salt manufacturing processes and techniques.

If a sea salt component is to be employed, such sea salt component can be derived from any suitable sea water source. Examples of useful sea salt components include Pacific sea salt, full-bodied sea salts, sea salts containing bitterns, sea salts containing trace minerals, sea salts having compositions similar to Pacific sea salt, course sea salt, fine sea salt, Dead Sea salt, and the like and mixtures thereof. The sea salt component useful in the present compositions may be made from one or more sources other than sea water. For example, mined salt may be used, together with a bitterns component and trace minerals to produce a “synthetic” sea salt component having a composition substantially similar to a sea salt component obtained from sea water. Such “synthetic” sea salt components are included within the scope of the present invention.

In one particularly useful embodiment, the sea salt component comprises a high quality sea salt product sold under the trademark SOUL OF THE SEA by H.K. Enterprise Group, Inc.

The present compositions may further include a bitterns component, for example, in an amount effective to provide the composition with a taste different from a substantially identical composition without the bitterns component. In one embodiment, the bitterns component is provided as a separate component, that is separate from the salt component. Alternately or in addition, the bitterns component, for example, at least a portion of the bitterns component, is part of the salt component. In a useful embodiment, the salt component comprises a sea salt component, and at least a portion of the bitterns component is part of the sea salt component.

The bitterns component may be present in an amount in a range of about 1.0 or about 2.0 to about 12 or about 20 or more milliliters per kilogram, or about 0.1 or about 0.2 to about 1.2 or about 2 or more teaspoons per pound, of salt component, for example, the sea salt component.

The present compositions may further comprise an aqueous component in an amount effective to facilitate the formation of the composition and/or provide at least a portion of a bitterns component to the composition and/or provide a taste to the composition. For example, the aqueous component may be an aqueous component selected from the group consisting of fresh water, sea water, concentrated sea water and mixtures thereof. In one embodiment, the aqueous component is present during the formation of the composition. The aqueous component may be present in an amount in a range of about 1.0 to about 15 or about 20 milliliters per kilogram, or about 0.1 to about 1.5 or about 2 teaspoons per pound, of the salt component.

In a useful embodiment, the present salt component, extract component-containing compositions further comprise a spice component, different from the salt component or sea salt component. Such composition is highly useful in combination with foods, such as vegetables, meats, sea food, poultry and the like and combinations thereof. Such compositions often comprise a combination of an extract component, as described herein, and a spice component. The combination of the extract component, for example and without limitation, the bamboo leaf extract component, the acai berry extract component and/or the lotus leaf extract component, and the spice component advantageously is present in an amount effective to provide at least one of (a) taste, (b) color (c) aroma, (d) a nutritional value and (e) a therapeutic value to the food into which the composition is placed. In one embodiment of the present invention, the extract component, for example, and without limitation, including bamboo leaf extract, has a sufficient amount of antioxidants to confer cardiovascular protective and anti-cancer properties to the food on or in which it is placed.

The spice component may include any individual spice or combination of two or more spices, for example, selected from spices which are conventionally used in foods and/or are known in the art. The spice component is included in an amount which may vary, for example, depending on the effect desired, the taste preference of the individual preparing or eating the food and the like factors. Examples of each spice components include, without limitation, one or more peppers, oregano, nutmeg, toasted sesame seeds, garlic, cilantro, ginger, mustard seeds, cayenne pepper, black pepper, onion, chilies, rosemary, basil, jalapeno, coriander, anise, cloves, and the like and mixtures thereof.

In one embodiment, the spice component comprises at least about 5% or about 10% or about 20% or about 40% by weight of the combination of the extract component and spice component. The spice component may include up to about 70% or about 80% or about 90% by weight or more of this combination.

In a further aspect of the invention, skin care compositions are provided. The present skin care compositions comprise a base oil component in an amount effective to provide at least one benefit to a skin region to which the composition is applied, and an antioxidant-containing botanical extract component, for example, as described elsewhere herein, combined with the base oil component.

In some embodiments, the antioxidant-containing botanical extract component of the present skin care composition is present in an amount effective to provide the composition with at least one of (a) an aroma, (b) a color and (c) an ability to provide a benefit or an enhanced benefit to a skin region of a human to which the composition is applied different from a substantially identical composition without the extract component. Such benefits may include, without limitation, providing new or enhanced one or more of therapeutic values, soothing benefits, restorative benefits, anti-aging benefits, emollient properties, exfoliating benefits and the like. In one embodiment, the extract component, for example, and without limitation, including bamboo leaf extract, which has a sufficient amount of antioxidants to provide highly useful anti-aging benefits, as well as other benefits, to the skin region.

The base oil component advantageously is present in an amount of at least about 20% or at least about 30% or at least about 40% or at least about 50% or more by weight of the composition. The extract component is present in an amount effective to provide the desired result, for example, to provide a new or enhanced benefit to a skin region to which the composition is applied. In one embodiment, the extract component is present in an amount in a range of about 0.05 or about 0.1 to about 0.5 or about 1 ml per 100 grams (about 1 or about 2 to about 10 or about 20 drops per 100 grams) of base oil component.

Any suitable base oil component may be employed in the present compositions provided it is effective in the present composition to provide the desired result and does not significantly detrimentally effect the composition or the person using the composition. The base oil component preferably comprises a material selected from the group consisting of beeswax, olive oil, avocado oil, jojoba oil, macadamia oil, sweet almond oil, aloe vera gel, rosemary essential oil, botanical oils, botanical extracts (for example, other than the extract components), and the like and mixtures thereof.

The composition may further comprise one or more additional components, different from the extract component, in amounts effective to provide the composition with one or more of (a) an aroma, (b) color, and (c) an ability to provide a benefit or an enhanced benefit to a skin region of a human to which the composition is applied different from a substantially identical composition without the additional component. Such benefits include, without limitation, therapeutic values, lubricating benefits, benefits occurring because of the surfactant properties of the additional component, and the like benefits.

Any suitable additional component may be included. For example, in some embodiments, the one or more additional components comprise a material selected from vitamins, lavender oil, lemon oil, other citrus oils, palm oil, palmarosa oil, tea tree oil and the like and mixtures thereof.

The one or more additional components are each present, it at all, in an amount in a range of about 0.02 or about 0.05 to about 0.5 milliliters per 100 grams (about 0.5 or about 1 to about 10 or more drops per 100 grams) of base oil component.

The present compositions may be substantially anhydrous, for example, is in the form of a balm. In other embodiments, the composition is in the form of a cream. Since the balm is anhydrous, it is absorbed slowly into skin. Therefore using such an anhydrous balm, in accordance with the present invention, allows the skin to be exposed to the composition longer than if a water-containing composition, for example, a water-containing cream, is used.

The composition may further comprise an aqueous component, which may even comprise a major amount by weight of the composition. Such a composition, including an aqueous component, may be in the form of cream, which is absorbed into the skin more rapidly and is less visible than an anhydrous balm, as discussed above.

In another aspect, the invention provides compositions useful in bathing at least a portion of a human body wherein the composition comprises a salt component and an antioxidant-containing botanical extract component, for example, as described elsewhere herein, combined with the salt component. The composition is effective in facilitating the bathing of at least a portion of a human body in water including an effective amount of the composition. The antioxidant-containing botanical extract component may be present in an amount effective to provide the composition with at least one of (a) an aroma, (b) a color, and (c) an ability to provide a benefit or an enhanced benefit to at least a portion of the human body bathed in water including an effective amount of the composition. Such benefit or enhanced benefit of the present composition may include providing an enhancement in one or more of the comfort, the quality of life, the physical well being, the physical health, the mental heath and the emotional health of the human whose body is bathed using the composition. Examples of such benefits to such human include, without limitation, providing new or enhanced nutritional values, therapeutic values, soothing benefits, restorative benefits, anti-aging benefits, skin exfoliating benefits, benefits occurring because of the surfactant properties of the extract component, and the like and combinations thereof.

Any suitable salt component may be used in the present compositions. Although the present compositions may include a salt component comprising pure or substantially pure sodium chloride, for example, mined or otherwise obtained or obtained by other than from sea water, the present compositions are very useful when the salt component comprises sea salt. In addition, the salt component may comprise a salt material selected from the group consisting of Epsom salt, sea salt, including course sea salt, fine sea salt and mixtures thereof, Dead Sea salt and the like and mixtures thereof.

The salt component may comprise a relatively large amount of the present composition, for example, a major amount, that is at least about 50%, by weight of the composition.

In one embodiment, the extract component is present in an amount in a range of about 0.2 or about 1.0 to about 17 or about 20 milliliters per kilogram of the salt component.

The present compositions may further comprise an additional component, different from the extract component, selected from essential oils, fragrant oils and mixtures thereof in an amount effective to provide the composition with at least one of (a) an aroma and (b) an ability to provide a benefit or an enhanced benefit to at least a portion of a human body bathed in water including an effective amount of the composition. Examples of useful additional components include, without limitation, ylang ylang essential oil, bergamot essential oil, eucalyptus essential oil, palmarosa essential oil, Australian niaouli essential oil, lemon essential oil, other citrus oils, vitamins, lavender essential oil, rosemary essential oil, mandarin essential oil, palm oil tea tree essential oil, fragrance oils and the like and mixtures thereof.

The additional component noted herein may be included in the present compositions in amounts over a large range of percentages. In one embodiment the additional component is present in an amount in a range of about 0.1 or about 0.2 to about 1.6 or about 2.0 or more milliliters per kilogram of the salt component.

Other components may be included in the present compositions useful for bathing. Such components include, for example and without limitation, an effervescence component in an amount effective when the composition is placed in water to provide effervescence to the water. For example, the effervescence component may comprise a combination of baking soda, soda and citric acid.

In a still further broad aspect of the invention, body and hair soap compositions are provided. Such compositions generally comprise a base oil component, a caustic component, for example, selected from a caustic soda component, a caustic potash component and the like and mixtures thereof, depending on the type of soap; a sea water component; and an antioxidant-containing botanical extract component, for example, as described elsewhere herein; and another or an additional component, different from the extract component, selected from essential oils, fragrance oils and mixtures thereof.

The extract component advantageously is present in an amount effective to provide the composition with at least one of (a) an aroma, (b) a color and (c) an ability to provide a benefit or an enhanced benefit to a body portion or a hair washed using the composition different from a substantially identical composition without the extract component. Examples of such benefits to such body portion or hair include providing new or enhanced cleansing, soothing, conditioning, restorative benefits, anti-aging benefits, exfoliating benefits and/or benefits obtained from the surfactant qualities or properties of the extract component.

The present compositions, for example, the present soap compositions, may include an amount of the extract component effective to provide the one or more benefits or enhanced benefits disclosed herein. In one embodiment, the extract component is present in an amount in a range of about 5 to about 100 milliliters per kilogram of the base oil component.

The base oil component advantageously comprises a major amount by weight of the composition. Any suitable base oil component may be employed. For example, the base oil component may comprise a material selected from olive oil, coconut oil, palm oil, jojoba oil, corn oil, avocado oil, other vegetable oils, other fruit oils and the like and mixtures thereof.

The base oil component may be present in an amount effective to provide the composition with at least one of (a) an aroma and (b) the ability to cleanse, condition, soothe, restore, emolliate and/or moisten the skin and/or hair of an individual bathing in water including an effective amount of the composition.

Each of the base oils imparts different characteristics to the soaps that it is present in, such as rich, stable, poor or no lather, mushiness, softness, medium hardness or hardness, cleansing, conditioning, soothing or restorative properties and short, medium or long tracing times, and the like properties. As used herein, the term “tracing time” refers to the time taken for the soap to start thickening during the manufacturing process. As used herein, the term “trace” refers to the time when the soap mixture begins to thicken. The more delicate ingredients are added as the soap mixture enters this “trace” state thereby avoiding the harsh chemical process taking place earlier.

The following table illustrates the characteristics that some base oils individually lend to soaps in which they are present.

| | Lather | Hardness | Soap | Tracing |
|---|---|---|---|---|
| Oil | from Soap | of Soap | Properties | time |
| Olive oil | Poor | Medium | Conditioning, | Medium |
| | | | soothing, | |
| | | | cleansing | |
| Coconut | Rich | Hard | Cleansing | short to |
| oil | | | | average |
| Palm oil | Stable | Hard | Cleansing | short to |
| | | | | very |
| | | | | short |
| Jojoba | None | Mushy | Restorative | average |
| oil | | | | to long |
| Corn oil | Poor | Soft | Conditioning | Long |
| Avocado | Stable | Soft | soothing, | average |
| oil | | | conditioning | to long |

The total base oil component is preferably present in an excess over the desired 1:1 base oil:caustic component weight ratio to provide superfatting. Superfatting percentage is the percentage by weight of total base oil component in excess of the caustic component over the actual amount of caustic component required to saponify the base oils. The superfatting percentage may vary over a relatively wide range. In one embodiment, the superfatting percentage is in the range of about 4 to about 6%. The superfatting base oils lend moisturizing and emollient properties to the soaps that they are present in and can be added at the trace stage.

As is evident to a person skilled in the art, the amount of caustic component is calculated based on the saponification values of the base oil components and the desired superfatting percentage.

The other or additional component of the present body and hair soap compositions may be present in an amount effective to provide the composition with at least one of (a) an aroma and (b) an ability to provide a benefit to the body portion or hair washed using the composition different from a substantially identical composition without the other or additional component.

The other or additional component may be present in an amount effective to provide the desired result. For example, the other or additional component may be present in an amount in a range of about 1.0 to about 10.0 milliliters for kilogram of the base oil component.

The other or additional component may comprise an oil selected from the group consisting of ylang ylang essential oil, bergamot essential oil, eucalyptus essential oil, palmarosa essential oil, Australian niaouli essential oil and the like and mixtures thereof. The other or additional component may be present in an amount in a range of about 1 to about 10 milliliters per kilogram of the total base oil component. The other or additional component may be added at trace.

In one embodiment, the present body and hair soap compositions further comprise a further component selected from a bitterns component, a seaweed component and mixtures thereof in an amount effective to provide the composition with at least one of (a) a different aroma than a composition without the further component and (b) an ability to provide a benefit or an enhanced benefit to a body portion or hair washed using the composition relative to a substantially identical composition without the further component.

In one useful embodiment, the further component is present in an amount in a range of about 5 to about 100 milliliters per kilogram of the base oil component.

The further component may be present in a range of about 5 to about 100 milliliters per kilogram of the total base oil component and is preferably added at trace.

The sea water component may be present at about 20 to about 40% by weight of the total base oil component.

The following non-limiting examples illustrate certain aspects of the present invention.

### EXAMPLES 1-6

A series of six (6) gourmet sea salt compositions are made using one of two sea salts, namely, Pacific sea salt or similar sea salt, and a full-bodied sea salt containing significant amounts of trace minerals, electrolytes and trace elements; bamboo leaf extract; and one or more of three aqueous components, namely, distilled or tap water, filtered and concentrated sea water, and a concentrated aqueous solution of bitterns. The bitterns concentration in such solution varies over a relatively wide range, and may be about 15% to about 45% by weight of the total solution. The compositions are prepared by mixing the ingredients and allowing the residual water to evaporate in appropriately sized, closed containers, over appropriate periods of time.

These compositions, which are referred to as Bamboo Jade sea salt compositions (and may have one or more other names), have the following compositions:

| | Ingredients | | | | | | | |
|---|---|---|---|---|---|---|---|---|
| Example | | | Bamboo Leaf | | | | | |
| Number | A | B | Extract | | C | | D | E |
| 1 | 2.27 kg | | 5 | mL | 24 | mL | 10 mL | |
| 2 | 2.0427 kg | .23 | 1.8 10 | mL | 1.8 6.5 | mL | | |
| 3 | 2.27 kg | | 1.8 10 | mL | | | | 630 mL |
| 4 | .23 | 2.0427 kg | .9 5 | mL | 10 | mL | | |
| 5 | | 2.27 kg | 5 | mL | 10 | mL | 5 mL | |
| 6 | | 2.27 kg | 5 | mL | | | 5 mL | 15 mL |

A = Pacific or similar sea salt
B = Full bodied sea salt
C = Aqueous bitterns solution
D = Distilled or Tap water
E = Filtered and concentrated sea water

### EXAMPLE 7-12

Each of the gourmet sea salt compositions produced in Examples 1-6 is advantageously used as an additive in food, such as, without limitation, rice, vegetables, meat and seafood, during food preparation and/or just prior to eating to impart a different aroma, color and taste to the foods. The compositions are also of therapeutic value to the human individuals consuming the foods, for example, because of the antioxidant and/or other advantageous properties of bamboo leaf extract. For example, the compositions are beneficial for cardiovascular health and for anti-cancer properties arising from the bamboo leaf extract.

### EXAMPLES 13-18

Examples 1-6 are repeated except that the bamboo leaf extract is replaced by acai berry extract.

### EXAMPLES 19-24

Each of the gourmet sea salt compositions produced in Examples 13-18 is advantageously used as an additive in food, such as, without limitation, rice, vegetables meat and seafood, during food preparation and/or just prior to eating to impart a different aroma, color and taste to the foods. The compositions are also of therapeutic value to the individuals consuming the foods, for example, because of the antioxidant and/or other advantageous properties of acai berry extract. For example, the compositions are beneficial for cardiovascular health and for anti-cancer properties arising from the acai berry extract.

### EXAMPLES 25-30

Examples 1-6 are repeated except that the bamboo leaf extract is replaced by lotus leaf extract.

### EXAMPLE 31-36

Each of the gourmet sea salt compositions produced in Examples 25-30 is advantageously used as an additive in food, such as, without limitation, rice, vegetables, meat and seafood, during food preparation and/or just prior to eating to impart a different aroma, color and taste to the foods. The compositions are also of therapeutic value to the individuals consuming the foods, for example, because of the antioxidant and/or other advantageous properties of lotus leaf extract. For example, the compositions are beneficial for cardiovascular health and for anti-cancer properties arising from the lotus leaf extract.

### EXAMPLE 37

A facial beauty cream, referred to as Hawaiian Angel, is prepared from the following ingredients.

| | | |
|---|---|---|
| Rose water | 123 | g |
| Baking soda | 1 | g |
| Sweet almond oil | 50 | g |
| Beeswax | 26 | g |
| Vitamin E | 0.1 | mL |
| Rosemary extract | 0.25 | mL |
| Bamboo leaf extract | 0.25 | mL |
| Bitterns | 0.25 | mL |

The method of preparation of the cream is within the skill in the art, for example, with reference to Remington: The Science and Practice of Pharmacy, Twenty First Edition (2005), by University of the Sciences in Philadelphia, published by Lippincott Williams & Wilkins, which is incorporated herein by reference in its entirety.

Briefly, rose water and baking soda are added in a suitable dish and stirred to obtain a clear aqueous solution. Beeswax and almond oil are then added to this solution and the mixture is heated using a water bath to melt the wax. The heating is discontinued after the wax is melted. After the mixture is cooled to below 120° F., the vitamin E, rosemary extract and bamboo leaf extract are added and mixed until homogenous.

### EXAMPLE 38

A female adult human with normal to dry facial skin applies the cream produced in Example 37 to her face on a daily basis in the morning and/or at night after facial cleansing. After 2 weeks the individual observes restoration of a youthful appearance and suppleness to the facial skin.

The bamboo leaf extract provides antioxidant properties and anti-aging benefits to the skin. In addition the almond oil and beeswax provide a pleasant aroma, and moisturize and/or emolliate the skin.

### EXAMPLE 39

Example 37 is repeated except that the bamboo leaf extract is replaced by acai berry extract.

### EXAMPLE 40

A female adult human with normal to dry facial skin applies the cream produced in Example 39 to her face on a daily basis in the morning and/or at night after facial cleansing. After 2 weeks the individual observes restoration of a youthful appearance and suppleness to the facial skin.

The acai berry extract provides antioxidant properties and anti-aging benefits to the skin.

In addition the sweet almond oil and beeswax provide a pleasant aroma, and moisturize and/or emolliate the skin.

### EXAMPLE 41

Example 37 is repeated except that the bamboo leaf extract is replaced by lotus leaf extract.

### EXAMPLE 42

A female adult human with normal to dry facial skin applies the cream produced in Example 41 to her face on a daily basis in the morning and/or at night after facial cleansing. After 2 weeks the individual observes restoration of a youthful appearance and suppleness to the facial skin.

The lotus leaf extract provides antioxidant properties and anti-aging benefits to the skin.

In addition the almond oil and beeswax provide a pleasant aroma, and moisturize and/or emolliate the skin.

### EXAMPLE 43

An anhydrous balm/gel, referred to as Hawaiian Phoenix, is prepared, for example, using conventional processing techniques as described in Remington, supra, from the following ingredients:

### Composition of balm/gel, called Hawaiian Phoenix

| | | |
|---|---|---|
| Beeswax | 20 | g |
| Olive oil | 75 | g |
| Avocado oil | 25 | g |
| Jojoba oil | 20 | g |
| Macadamia oil | 20 | g |
| Rosemary extract | 1 | g |

Oils added below 120° F.

| | | |
|---|---|---|
| Vitamin E | 0.1 | mL |
| Lavender oil | 0.25 | mL |
| Rosemary extract | 0.25 | mL |
| Lemon oil | 0.25 | mL |
| Palmarosa oil | 0.25 | mL |
| Tea tree oil | 0.25 | mL |
| Bamboo leaf extract | 0.25 | mL |
| Bitterns | 0.25 | mL |

Briefly, the beeswax is heated using a water bath to melt the wax and the olive oil, avocado oil, jojoba oil, macadamia oil and rosemary extract are added mixed until homogenous. The heating is then discontinued. After the mixture is cooled to below 120° F., the vitamin E, lavender oil, rosemary extract, lemon oil, palmarosa oil, tea tree oil and bamboo leaf extract are added and mixed until homogenous.

### EXAMPLE 44

A male adult human with dry and chapped skin on hands, feet, elbows and other skin areas applies the balm in Example 43 to such skin areas on a daily basis in the morning and/or at night after cleansing the affected areas. After 2 weeks the individual reports healing of the skin areas and restoration of suppleness and moistness to the skin areas where the balm is applied.

The bamboo leaf extract component provides antioxidant properties and anti-aging benefits to the skin areas. In addition the oils in the balm provide a pleasant aroma, and moisturize and/or emolliate the skin areas. The anhydrous nature of the balm helps to slow absorption so that the dry and chapped skin is benefited more from longer exposure to the balm.

### EXAMPLE 45

Example 43 is repeated except that the bamboo leaf extract is replaced by acai berry extract.

### EXAMPLE 46

A male adult human with dry and chapped skin on hands, feet, elbows and other skin areas applies the balm in Example 45 to such skin areas on a daily basis in the morning and/or at night after cleansing the affected areas. After 2 weeks the individual reports healing of the skin areas and restoration of suppleness and moistness to the skin areas where the balm is applied.

The acai berry extract component provides antioxidant properties and anti-aging benefits to the skin areas. In addition the oils in the balm provide a pleasant aroma, and moisturize and/or emolliate the skin areas. The anhydrous nature of the balm helps to slow absorption so that the dry and chapped skin is benefited more from longer exposure to the balm.

### EXAMPLE 47

Example 43 is repeated except that the bamboo leaf extract is replaced by lotus leaf extract.

### EXAMPLE 48

A male adult human with dry and chapped skin on hands, feet, elbows and other skin areas applies the balm in Example 47 to such skin areas on a daily basis in the morning and/or at night after cleansing the affected areas. After 2 weeks the individual reports healing of the skin areas and restoration of suppleness and moistness to the skin areas where the balm is applied.

The lotus leaf extract component provides antioxidant properties and anti-aging benefits to the skin areas. In addition the oils in the balm provide a pleasant aroma, and moisturize and/or emolliate the skin areas. The anhydrous nature of the balm helps to slow absorption so that the dry and chapped skin is benefited more from longer exposure to the balm.

### EXAMPLES 49-54

A series of six (6) sea salt crystal compositions containing bamboo leaf extract are prepared. Each of the preparations is made by blending all the ingredients to homogeneity. These compositions are as follows:

Example 49, referred to as Hawaiian Green Goddess

| | | |
|---|---|---|
| Epsom salt | 290 | g |
| Sea salt | 580 | g |
| Ylang Ylang essential oil | 0.5 | mL |
| Bergamot essential oil | 0.25 | mL |
| Eucalyptus essential oil | 1 | mL |
| Bamboo leaf extract | 1.25 | mL |

Example 50, referred to as Hawaiian Jungle mist

| | | |
|---|---|---|
| Coarse sea salt | 580 | g |
| Citric acid | 145 | g |
| Baking soda | 110 | g |
| Palmarosa essential oil | 0.5 | mL |
| Australian niaouli essential oil | 0.5 | mL |
| Lemon essential oil | 0.5 | mL |
| Bamboo leaf extract | 5 | mL |

Example 51, referred to as Hawaiian Jade Moon

| | | |
|---|---|---|
| Coarse sea salt | 580 | g |
| Fine sea salt | 290 | g |
| Ylang Ylang essential oil | 0.75 | mL |
| Lavender essential oil | 0.5 | mL |
| Rosemary essential oil | 0.5 | mL |
| Bamboo leaf extract | 1.25 | mL |

Example 52, referred to as Chinese Princess

| | | |
|---|---|---|
| Coarse sea salt | 580 | g |
| Fine sea salt | 145 | g |
| Epsom salt | 145 | g |
| Ylang Ylang essential oil | 0.75 | mL |
| Lavender essential oil | 0.5 | mL |
| Mandarin essential oil | 0.5 | mL |
| Bamboo leaf extract | 1.25 | mL |

Example 53, referred to as to as China Pearl

| | | |
|---|---|---|
| Coarse sea salt | 580 | g |
| Fine sea salt | 145 | g |
| Dead Sea salt | 145 | g |
| Ylang Ylang essential oil | 0.75 | mL |
| Lavender essential oil | 0.5 | mL |
| Lemon Tea tree essential oil | 0.5 | mL |
| Bamboo leaf extract | 1.25 | mL |
| Lotus leaf extract | 1.25 | mL |

Example 54, referred to as Time Out Please

| | | |
|---|---|---|
| Coarse sea salt | 290 | g |
| Epsom salt | 72.5 | g |
| Dead Sea salt | 72.5 | g |
| Ylang Ylang essential oil | 0.25 | mL |
| Lavender essential oil | 0.25 | mL |
| Lemon Tea tree essential oil | 0.25 | mL |
| Bamboo leaf extract | 0.625 | mL |

### EXAMPLE 55-59

Each of the sea salt crystal compositions produced in Examples 49-53 are used as bath salts to provide an adult human individual with a restorative and invigorating bathing experience. The bamboo leaf extract provides anti-aging and skin rejuvenating properties to the compositions. In addition the essential oils provide a pleasant aroma, and moisturize and/or emolliate the skin of the individual bathing in water containing such compositions.

### EXAMPLE 60

The sea salt crystal composition produced in Example 54 is added to about 1.4 liters of hot water to provide a foot soak for the feet of an adult human individual, which foot soak is effective to relieve and rejuvenate an individual with tired and overworked feet.

### EXAMPLES 61-66

Examples 49-54 are repeated except that the bamboo leaf extract is replaced by acai berry extract.

### EXAMPLES 67-71

Each of the sea salt crystal compositions produced in Examples 61-65 are used as bath salts to provide an adult human individual with a restorative and invigorating bathing experience. The acai berry extract provides anti-aging and skin rejuvenating properties to the compositions. In addition the essential oils provide a pleasant aroma, and moisturize and/or emolliate the skin of the individual bathing in water containing such compositions.

### EXAMPLE 72

The sea salt crystal composition produced in Example 66 is added to about 1.4 liters of hot water to provide a foot soak for the feet of an adult human individual, which foot soak is effective to relieve and rejuvenate an individual with tired and overworked feet.

### EXAMPLES 73-78

Examples 49-54 are repeated except that the bamboo leaf extract is replaced by lotus leaf extract.

### EXAMPLES 79-83

Each of the sea salt crystal compositions produced in Examples 73-77 are used as bath salts to provide an adult human individual with a restorative and invigorating bathing experience. The lotus leaf extract provides anti-aging and skin rejuvenating properties to the compositions. In addition the essential oils provide a pleasant aroma, and moisturize and/or emolliate the skin of the individual bathing in water containing such compositions.

### EXAMPLE 84

The sea salt crystal composition produced in Example 78 is added to about 1.4 liters of hot water to provide a foot soak for the feet of an adult human individual, which foot soak is effective to relieve and rejuvenate an individual with tired and overworked feet.

### EXAMPLES 85-86

Two kinds of soaps containing bamboo leaf extract and/or lotus leaf extract in addition to bitterns are made with the compositions described below.

The soaps are manufactured by a conventional process known to the person skilled in the art. Briefly, the base oil component, caustic soda and filtered sea water are first heated to 110° F. with stirring/blending. The superfatting percentage of base oils used is about 4.2%. At trace, when the soap starts thickening, the essential oils, bamboo leaf extract and/or lotus leaf extract and/or seaweed and/or bitterns are blended in. The mixture is poured into appropriately sized molds, cured, dried and cut into bars. The batch yield of the soaps described here is approximately 75% and the batch size described below yields approximately ten bars that are 3.8 ounces in weight and approximately six bars that are 6 ounces in weight after curing and drying.

The soap of Example 85, referred to as Hawaiian Bamboo Jade body and hair soap, is prepared from the following ingredients:

| | | | | Amount of |
|---|---|---|---|---|
| | | | | caustic soda |
| Ingredients | | | | in grams |
| added | Amount | Amount | | required for |
| before | in | in | Saponification | saponification |
| trace | grams | ounces | value | of base oil |
| Olive oil | 200 | 7.1 | 0.134 | 26.8 |
| Coconut oil | 290 | 10.2 | 0.187 | 54.23 |
| Palm oil | 70 | 2.5 | 0.141 | 9.87 |
| Jojoba oil | 40 | 1.4 | 0.059 | 2.36 |
| Corn oil | 200 | 7.1 | 0.136 | 27.20 |
| Avocado oil | 200 | 7.1 | 0.133 | 26.60 |
| (cold | | | | |
| process, | | | | |
| unrefined) | | | | |
| Total Base | 1000 | 35.3 | n/a | 147.06 |
| Oils | | | | |
| Caustic | 141.1 | 5.0 | n/a | |
| Soda | | | | |
| Filtered | 334 | 11.8 | | |
| sea water | | | | |

| Ingredients | | Amount | |
|---|---|---|---|
| added at | Amount | in | Amount in |
| trace | in mL | drops | fluid ounces |
| Ylang ylang | 6 | 117 | |
| oil | | | |
| Bergamot | 4 | 78 | |
| oil | | | |
| Palmarosa | 4 | 78 | |
| essential | | | |
| oil | | | |
| Australian | 4 | 78 | |
| niaouli | | | |
| essential | | | |
| oil | | | |
| Total | 18 | 351 | 0.61 |
| essential | | | |
| oils | | | |
| Bamboo leaf | 20 | | 0.68 |
| extract | | | |
| Seaweed | 20 | | 0.68 |
| Bitterns | 20 | | 0.68 |

The soap of Example 86, referred to as Hawaiian China Pearl body and hair soap, is prepared from the following ingredients:

| | | | | Amount of |
|---|---|---|---|---|
| | | | | caustic soda |
| Ingredients | | | | in grams |
| added | Amount | Amount | | required for |
| before | in | in | Saponification | saponification |
| trace | grams | ounces | value | of base oil |
| Olive oil | 600 | 21.2 | 0.134 | 80.4 |
| | | 7.1 | | |
| Coconut oil | 290 | 10.2 | 0.187 | 54.23 |
| Palm oil | 70 | 2.5 | 0.141 | 9.87 |
| Jojoba oil | 40 | 1.4 | 0.059 | 2.36 |
| Total Base | 1000 | 35.3 | n/a | 146.86 |
| oils | | | | |
| Caustic | 140.9 | 5.0 | n/a | |
| Soda | | | | |
| Filtered | 334 | 11.8 | | |
| sea water | | | | |

| Ingredients | | Amount | |
|---|---|---|---|
| added at | Amount | in | Amount in |
| trace | in mL | drops | fluid ounces |
| Ylang ylang | 6 | 117 | |
| (oil) | | | |
| Bergamot | 4 | 78 | |
| oil | | | |
| Palmarosa | 4 | 78 | |
| essential | | | |
| oil | | | |
| Total | 14 | 273 | 0.47 |
| essential | | | |
| oils | | | |
| Bamboo leaf | 20 | | 0.68 |
| extract | | | |
| Chinese | 20 | | 0.68 |
| lotus leaf | | | |
| extract | | | |
| Bitterns | 20 | | 0.68 |

### EXAMPLES 87-88

The hair and body soaps in Examples 85 and 86 are used for cleansing, soothing, conditioning and restoration of skin and/or hair of a male human individual. The bamboo leaf extract and/or lotus leaf extract in the soaps provide antioxidant and anti-aging benefits, so that the individual, with daily use of the soap for a period of one month, has more healthy hair and skin relative to prior to using the soap.

### EXAMPLE 89-90

Two (2) soaps are made having substantially similar compositions as the soaps of Examples 85 and 86 except that the bamboo leaf extract in the soap of Example 85 is replaced by acai berry extract (in the soap of Example 89), and the lotus leaf extract in the soap of Example 86 is replaced by acai berry extract (in the soap of Example 90).

### EXAMPLE 91-92

The hair and body soaps in Examples 89 and 90 are used for cleansing, soothing, conditioning and restoration of skin and/or hair of a male human individual. The bamboo leaf extract and/or acai berry extract in the soaps provide antioxidant and anti-aging benefits, so that the individual, with daily use of the soap for a period of one month, has more healthy hair and skin relative to prior to using the soap.

### EXAMPLE 93

A spice mixture, referred to as Hunan Spice, is combined with the sea salt compositions of Examples 1-6 to provide a spice composition that provides at least one of (a) a different taste, (b) a different color and/or (c) a different aroma, relative to the sea salt composition without the spice mixture, to the food to which the composition is added.

The spice composition is prepared by mixing toasted sesame seeds, dried garlic flakes, ground cilantro, ground ginger, mustard seeds, cayenne pepper, Bamboo Jade Sea Salt and ground black pepper together in individual amounts which may vary, for example, depending on the effect desired, the taste preference of the individual preparing or eating the food and the like factors.

The bamboo leaf extract component present in the spice composition provides substantially similar benefits as it does in the sea salt compositions of Examples 1-6.

### EXAMPLE 94

The spice composition prepared in Example 93 is highly useful in combination with foods, such as vegetables, meats, sea food, poultry and the like and combinations thereof. The following is an example of a food recipe with including such spice composition that serves about four people.

About 14 grams of peanut oil is heated in a wok or large skillet. About one pound of cubed, boneless chicken is then added and seasoned with about 14 or more grams of the spice composition and stir fried over moderate heat until golden brown, then placed on a platter. About 14 grams of peanut oil is heated in a wok and about 200 g of diced scallions, about 200 g of snow peas, about 200 g of bean sprouts, about 200 g of chopped bok choy and about 200 g of chopped Asian cabbage are added, seasoned with about 14 grams or more of the spice composition and stir fried on high heat until the vegetables are cooked but firm/al dente, for about 5 minutes. About 200 g of sliced mushrooms and the chicken cooked above are added and cooked further for about a minute. This can be served with rice and makes for about 4 servings.

The vegetables can be substituted with other Asian vegetables and combinations thereof and the chicken can be substituted with other meats, sea food or poultry and combinations thereof. The bamboo leaf extract component present in the spice composition provides antioxidant benefits, and therefore has cardiovascular protective and anti-cancer properties.

### EXAMPLES 95-96

Examples 93 and 94 are repeated except that the sea salt compositions of Examples 13-18 are used instead of the sea salt compositions of Examples 1-6. Substantially similar results are obtained with modified or different flavors.

### EXAMPLES 97-98

Examples 93 and 94 are repeated except that the sea salt compositions of Examples 25-30 are used instead of the sea salt compositions of Examples 1-6. Substantially similar results are obtained with modified or different favors.

Compositions and methods of using such compositions similar to the compositions and use methods of Examples 1-98 are produced and practiced using the other extract components described herein in place of the bamboo leaf extract/the acai berry extract/the lotus leaf extract of Examples 1-98. Useful results are obtained, keeping in mind that compositions including the other extract components have different characteristics and properties, because of the inclusion of the different extract components.

While this invention has been described with respect to various specific examples and embodiments, it is to be understood that the invention is not limited thereto and that it can be variously practiced within the scope of the following claims.

1. A salt composition comprising:
a sea salt component; and
an antioxidant-containing botanical extract component combined with the sea salt component.

2. The composition of claim 1, wherein the extract component is present in an amount effective to provide the composition with one or more of (a) a taste, (b) an aroma, (c) a color, (d) a nutritional value and (e) a therapeutic value different from a substantially identical composition without the extract component.

3. The composition of claim 1, wherein the extract component is selected from the group consisting of a bamboo leaf extract component, an acai berry extract component, a lotus leaf extract component, a pitaya extract component, a green tea extract component, a red tea extract component, a Reishi mushroom extract component, a Shitake mushroom extract component, a licorice root extract component, a burdock root extract component, a soy extract component, a carrot extract component, a tomato extract component, a broccoli extract component, a Chlorella extract component, a wheatgrass extract component, a Laminaria Digitata extract component, an alfalfa extract component, a Rose Hips extract component, a ginger extract component, a clove extract component, a cinnamon extract component, an oregano extract component, a thyme extract component, a fennel extract component, a sage extract component, a rosemary extract component, a turmeric extract component, a garlic extract component, an onion extract component, a ginseng extract component, an Astragalus extract component, a Schizandra extract component, a Fenugreek extract component, and mixtures thereof.

4. (canceled)

5. (canceled)

6. The composition of claim 1, which includes a bitterns component in an amount effective to provide the composition with a taste different from a substantially identical composition without the bitterns component.

7. (canceled)

8. (canceled)

9. (canceled)

10. (canceled)

11. The composition of claim 1, which further comprises an aqueous component in an amount effective to perform one or more of (1) facilitate the formation of the composition; (2) provide a bitterns component to the composition; and (3) provide a taste to the composition.

12. (canceled)

13. The composition of claim 1, which further comprises a spice component in an amount effective to provide the composition with one or more of (a) a taste, (b) a color, and (c) an aroma different from a substantially identical composition without the spice component.

14. A skin care composition comprising
a base oil component in an amount effective to provide at least one benefit to a skin region of a human to which the composition is applied; and
an antioxidant-containing botanical extract component combined with the base oil component.

15. The composition of claim 14, wherein the extract component is present in an amount effective to provide the composition with at least one of (a) an aroma, (b) a color, and (c) an ability to provide a benefit to a skin region of a human to which the composition is applied different from a substantially identical composition without the extract component.

16. The composition of claim 14, wherein the extract component is selected from the group consisting of a bamboo leaf extract component, an acai berry extract component, a lotus leaf extract component, a pitaya extract component, a green tea extract component, a red tea extract component, a Reishi mushroom extract component, a Shitake mushroom extract component, a licorice root extract component, a burdock root extract component, a soy extract component, a carrot extract component, a tomato extract component, a broccoli extract component, a Chlorella extract component, a wheatgrass extract component, a Laminaria Digitata extract component, an alfalfa extract component, a Rose Hips extract component, a ginger extract component, a clove extract component, a cinnamon extract component, an oregano extract component, a thyme extract component, a fennel extract component, a sage extract component, a rosemary extract component, a turmeric extract component, a garlic extract component, an onion extract component, a ginseng extract component, an Astragalus extract component, a Schizandra extract component, a Fenugreek extract component, and mixtures thereof.

17. The composition of claims 14, wherein the extract component is selected from the group consisting of a bamboo leaf extract component, an acai berry extract component, a lotus leaf extract component and mixtures thereof.

18. (canceled)

19. (canceled)

20. The composition of claim 14, wherein the base oil component comprises a material selected from the group consisting of beeswax, olive oil, avocado oil, jojoba oil, macadamia oil, rosemary essential oil, sweet almond oil and mixtures thereof.

21. The composition of claim 14, which further comprises an additional component present in an amount effective to provide the composition with one or more of (a) an aroma, (b) a color and (c) an ability to provide a benefit to a skin region of a human to which the composition is applied different from a substantially identical composition without the additional component.

22. The composition of claim 21, wherein the additional component is selected from the group consisting of vitamins, lavender oil, rosemary essential oil, lemon oil, other citrus oils, palm oil, palmarosa oil, tea tree oil, fragrance oils and mixtures thereof.

23. (canceled)

24. The composition of claim 14, which is substantially anhydrous.

25. The composition of claim 14, which is in the form of a balm.

26. The composition of claim 14, which further comprises an aqueous component.

27. (canceled)

28. The composition of claim 14, which is in the form of a cream.

29. A composition useful in bathing at least a portion of a human body, the composition comprising:
a salt component; and
an antioxidant-containing botanical extract component combined with the salt component, the composition being effective in facilitating the bathing of at least a portion of a human body in water including an effective amount of the composition.

30. The composition of claim 29, wherein the extract component is present in an amount effective to provide the composition with one or more of (a) an aroma, (b) a color and (c) an ability to provide a benefit to at least a portion of a human body bathed in water including an effective amount of the composition different from a substantially identical composition without the extract component.

31. The composition of claim 29, wherein the extract component is selected from the group consisting of a bamboo leaf extract component, an acai berry extract component, a lotus leaf extract component, a pitaya extract component, a green tea extract component, a red tea extract component, a Reishi mushroom extract component, a Shitake mushroom extract component, a licorice root extract component, a burdock root extract component, a soy extract component, a carrot extract component, a tomato extract component, a broccoli extract component, a Chlorella extract component, a wheatgrass extract component, a Laminaria Digitata extract component, an alfalfa extract component, a Rose Hips extract component, a ginger extract component, a clove extract component, a cinnamon extract component, an oregano extract component, a thyme extract component, a fennel extract component, a sage extract component, a rosemary extract component, a turmeric extract component, a garlic extract component, an onion extract component, a ginseng extract component, an Astragalus extract component, a Schizandra extract component, a Fenugreek extract component, and mixtures thereof.

32. (canceled)

33. (canceled)

34. The composition of claim 29, which further comprises an additional component selected from the group consisting of essential oils, fragrance oils and mixtures thereof in an amount effective to provide the composition with at least one of (a) an aroma, and (b) an ability to provide a benefit or an enhanced benefit to at least a portion of a human body bathed in water including an effective amount of the composition different from a substantially identical composition without the additional component.

35. (canceled)

36. (canceled)

37. The composition of claim 29, wherein the salt component comprises sea salt.

38. (canceled)

39. (canceled)

40. (canceled)

41. A body and hair soap composition comprising:
a base oil component;
a caustic component;
a sea water component;
an antioxidant-containing botanical extract component selected from the group consisting of a bamboo leaf extract component; and
an additional component selected from the group consisting of essential oils, fragrance oils and mixtures thereof.

42. (canceled)

43. The composition of claim 41, wherein the extract component is present in an amount effective to provide the composition with one or more of (a) an aroma, (b) a color, and (c) an ability to provide a benefit to a body portion or hair washed using the composition different from a substantially identical composition without the extract component.

44. The composition of claim 41, wherein the extract component is selected from the group consisting of a bamboo leaf extract component, an acai berry extract component, a lotus leaf extract component, a pitaya extract component, a green tea extract component, a red tea extract component, a Reishi mushroom extract component, a Shitake mushroom extract component, a licorice root extract component, a burdock root extract component, a soy extract component, a carrot extract component, a tomato extract component, a broccoli extract component, a Chlorella extract component, a wheatgrass extract component, a Laminaria Digitata extract component, an alfalfa extract component, a Rose Hips extract component, a ginger extract component, a clove extract component, a cinnamon extract component, an oregano extract component, a thyme extract component, a fennel extract component, a sage extract component, a rosemary extract component, a turmeric extract component, a garlic extract component, an onion extract component, a ginseng extract component, an Astragalus extract component, a Schizandra extract component, a Fenugreek extract component, and mixtures thereof.

45. (canceled)

46. (canceled)

47. (canceled)

48. The composition of claim 41, the additional component is present in an amount effective to provide the composition with one or more of (a) an aroma and (b) an ability to provide a benefit to a body portion or hair washed using the composition different from a substantially identical composition without the additional component.

49. (canceled)

50. (canceled)

51. The composition of claim 41, which further comprises a further component selected from the group consisting of a bitterns component, a seaweed component and mixtures thereof in an amount effective to provide the composition with one or more of (a) an aroma and (b) an ability to provide a benefit to a body portion or hair washed using the composition different from a substantially identical composition without the further component.

52. (canceled)
